(19) United States
(12) Patent Application Publication
(10) Pub. No.: US 2010/0047122 A1
(43) Pub. Date: Feb. 25, 2010

(54) SYSTEM AND METHOD FOR AUTOMATED STERILE SAMPLING OF FLUID FROM A VESSEL

(75) Inventor: George E. Barringer, JR., West Groton, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: GROTON BIOSYSTEMS, LLC, Boxborough, MA (US)

(21) Appl. No.: 12/490,960

(22) Filed: Jun. 24, 2009

Related U.S. Application Data
(60) Provisional application No. 61/133,209, filed on Jun. 25, 2008.

(51) Int. Cl. G01N 1/10; C12M 1/26; G01N 1/18; A61L 2/07; A61L 2/24

(52) U.S. Cl. 422/26; 422/292

(57) ABSTRACT

A sampling system includes a steam source, a steam valve connected the steam source, a sampling valve connected to the fluid sample source, an isolation valve, a processing module, a drain valve, a drain, and a controller. The controller to passes cleaning fluid from the processing module through the isolation valve to the drain, passes steam through the steam valve, sampling valve, and isolation valve to the drain for a duration sufficient to sterilize the sampling valve, the isolation valve, and a fluid path therebetween, and passes fluid samples from the fluid sample source through the sampling valve and isolation valve to the processing module. The system and a method described delivers safer, more consistent sampling, while reducing the risk of contamination during extraction of a sample from a vessel and minimizing waste of the sample.

### RELATED APPLICATION(S)

This application claims the benefit of U.S. Provisional Application No. 61/133,209, filed on Jun. 25, 2008. The entire teachings of the above application are incorporated herein by reference.

### BACKGROUND OF THE INVENTION

In a bioreactor process, maintaining a contamination-free environment is key. Whenever a bioprocess system is exposed to the external environment, it faces the risk of contamination by viruses, micro-organisms, and chemicals. Typical bioprocesses involve batch bioreactors where cells are cultured and harvested over a period of time ranging from minutes to days. After a batch is harvested, the reactor vessel is sterilized in preparation for the next batch process. For small volume reactors, the entire reactor system can be placed in an autoclave and completely sterilized. For example, reactors that are about 5 liters or less typically are made of glass and are sterilized in an autoclave. However, large volume reactors, such as those that are about 5 liters or more are typically too large to be placed in an autoclave, and must therefore be sterilized using Clean-in-Place (CIP) and Steam-in-Place (SIP) methods. CIP and SIP are methods used in the pharmaceutical and food industries for the in-line sterilization of processing equipment, including vessels, valves, process lines, and filter assemblies. These methods are used to achieve sterility or a certain level of sanitation required by regulation for a particular process.

In many cases, bioreactor processes do not lend themselves easily to in-situ analysis of the batch. Instead, samples must be physically extracted from the process and examined and manipulated outside the vessel, thereby exposing the entire batch to the external environment and the possibility of contamination. Since loss of a sample run or contamination of the process can have extremely expensive ramifications, it is important to obtain a sample without causing contamination. Furthermore, to minimize waste of the batch material, it is desirable to extract a sample only in the amount necessary for processing and analysis.

Many reactors are equipped with a sampling valve whereby the contents of the reactor may be extracted. Referring to FIG. 1, a sampling valve 3 of a fluid sample source 1 is connected to capped input and output ports, 7 and 9, respectively. The typical process for extracting a sample from a reactor involves manual operation. A human operator first opens the capped input port 7 and drain output port 9. The operator then uses a tri-clamp to connect a steam source 5 to the input port 7 and a steam drain 10 to the drain output port 9. The operator opens a steam valve 13 to permit steam from steam source 5 to pass for a specified amount of time through the input port 7, sampling valve 3, and drain output port 9 and to exit to the drain. Once the sampling valve 3 is sufficiently sterilized, the operator terminates the steam operation by closing the steam valve 13. The operator then disconnects drain output port 9 from the drain and manually draws a sample from the reactor 1 through the sample valve 3 and drain output port 9 into a container. After the sample has been extracted, the operator can optionally sterilize the system again by reconnecting the drain output port 9 to the drain and opening steam valve 13 to run steam through the components, as described above. Finally, the operator disconnects the drain output port 9 from the drain and disconnects the steam input port 7 from the steam source, recapping both ports.

The described process is susceptible to the introduction of contamination in various ways; the sterilizing and sampling processes are always subject to the possibility of human error, and the routine connecting and disconnecting of the lines brings constant exposure of the system to contamination from the external environment. In some instances, the sample may leak from the sampling valve, unnecessarily wasting portions of the batch and, if the batch material is biohazardous, possibly injuring the operator. In addition, the process places the operator at risk of burn injuries during the steam operation.

### SUMMARY OF THE INVENTION

What is needed is an improved system and method for acquiring samples from a bioreactor that is safer, more consistent, and less susceptible to contamination.

In one aspect, provided is an automatic sterile sampling system for sampling fluid samples from a sample source and providing the sample to a processing system, comprising a steam valve to receive steam from a steam source, a fluid sample source, a processing system to process fluid samples from the fluid sample source and comprising a cleaning fluid source, a sampling valve to receive fluid samples from the fluid sample source and connected to receive the steam from the steam valve, an isolation valve to pass steam from the sampling valve to a drain, pass fluid samples from the sampling valve to the processing system, and pass cleaning fluid from the processing system to the drain, and a controller configured to control the valves to control the flow of the steam, the fluid sample, and the cleaning fluid.

In another aspect, provided is a method for automatic aseptic sampling from a fluid sample source, comprising the steps of providing a steam source, a steam valve connected the steam source, a sampling valve connected to the fluid sample source, an isolation valve, a processing module, a drain valve, a drain, and a controller; and employing the controller to pass cleaning fluid from the processing module through the isolation valve to the drain, pass steam through the steam valve, sampling valve, and isolation valve to the drain for a duration sufficient to sterilize the sampling valve, the isolation valve, and a fluid path therebetween, and pass fluid sample from the fluid sample source through the sampling valve and isolation valve to the processing module.

Thus provided are a system and a method that delivers safer, more consistent sampling, while reducing the risk of contamination during extraction of a sample from a vessel. Waste of the sample can also be minimized.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1 is a drawing of a manually operated sampling system connected to a bioreactor;

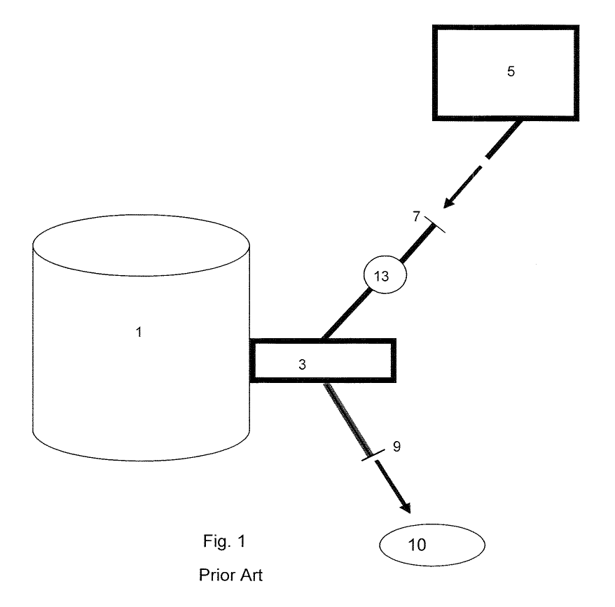

FIG. 2 is a drawing of the automated system at rest;

FIG. 3 is a drawing of the isolation valve and drain valve functioning in cooperation during a sterilizing operation;

FIG. 4 is a drawing of the isolation valve and drain valve functioning in cooperation during a sampling operation;

FIG. 5a is a drawing of the isolation valve and drain valve functioning in cooperation during a first part of a sanitizing operation;

FIG. 5b is a drawing of the isolation valve and drain valve functioning in cooperation during a second part of a sanitizing operation;

FIG. 6 is a drawing of the control valve system for the isolation valve;

FIG. 7 is a drawing of the automated system at rest, including a controller that is separate from the processing system;

FIG. 8a is a drawing of the automated system during a first part of a sanitizing operation;

FIG. 8b is a drawing of the automated system during a second part of a sanitizing operation;

FIG. 9 is a drawing of the automated system during a sterilizing operation;

FIG. 10 is a drawing of the sampling valve during a sterilizing operation;

FIG. 11 is a drawing of the sampling valve during a sampling operation;

FIG. 12 is a drawing of the automated system during a sterilizing operation, including sterilizing a portion of the sample transfer channel;

FIG. 13 is a drawing of the automated system during a cooling operation;

FIG. 14 is a drawing of the automated system during a sampling operation; and

FIG. 15 is a drawing of the system during a manual sampling operation.

### DETAILED DESCRIPTION OF THE INVENTION

A description of example embodiments of the invention follows. The embodiments provide an automated system and method for extracting a sample from a batch reactor while maintaining sterility of the key components through which the sample is extracted. The invention is not limited to sampling from a bioreactor, but rather can be applied to the aseptic sampling of any vessel containing a fluid. The system employs a series of pneumatically actuated valves to control the flow of steam, fluid sample, cleaning fluid, and optionally air through the system at specified times and includes a connection whereby a fluid sample is routed from the bioreactor vessel to a downstream processing system. As used herein, the term “valve” refers to a single valve or system of valving that achieves a particular flow configuration.

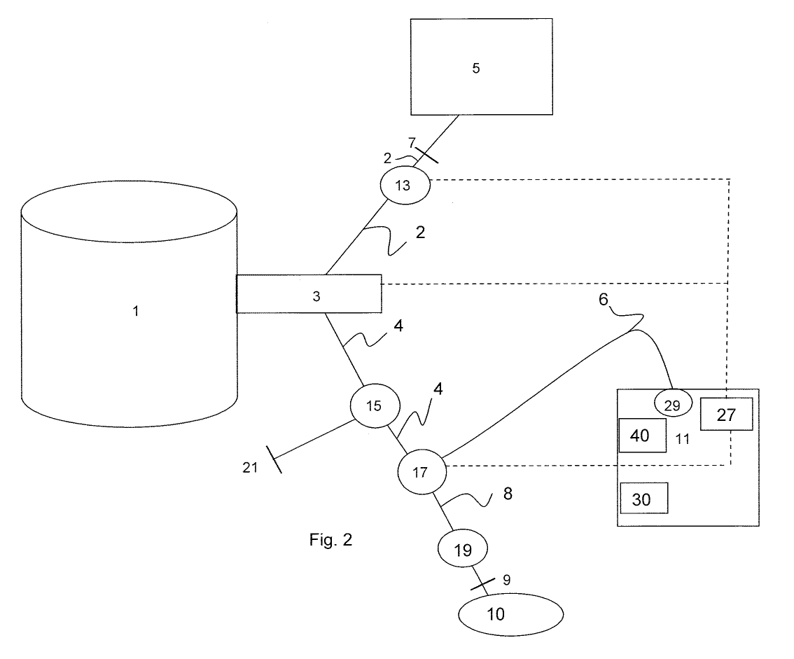

Referring to FIG. 2, the automated sampling system includes a steam channel 2 having a steam input port 7 that is semi-permanently connected to a steam source 5. The system also includes a drain channel 8 having a drain output port 9 that is semi-permanently connected to a drain. As used herein, the term “semi-permanent” refers to a connection between components that is maintained during normal operation and is ordinarily not disconnected unless system maintenance is required. Unlike previous sampling systems, the entire system is connected at all times during operation of the reactor, thereby minimizing the opportunity for exposure of the process to the external environment and reducing the likelihood of an incomplete connection between the system components.

Returning to FIG. 2, the system further includes a steam valve 13, a sampling valve 3, an isolation valve 17, an optional manual sampling valve 15, and a drain valve 19.

Steam valve 13 controls the flow of steam through a steam channel 2. Steam valve 13 is typically a diaphragm valve, such as GEMÜ® Type 650/015/D80415A0-1537, which is a ½ inch two-port pneumatically actuated sanitary valve. When steam valve 13 is open, steam is allowed to pass through steam channel 2 to sampling valve 3.

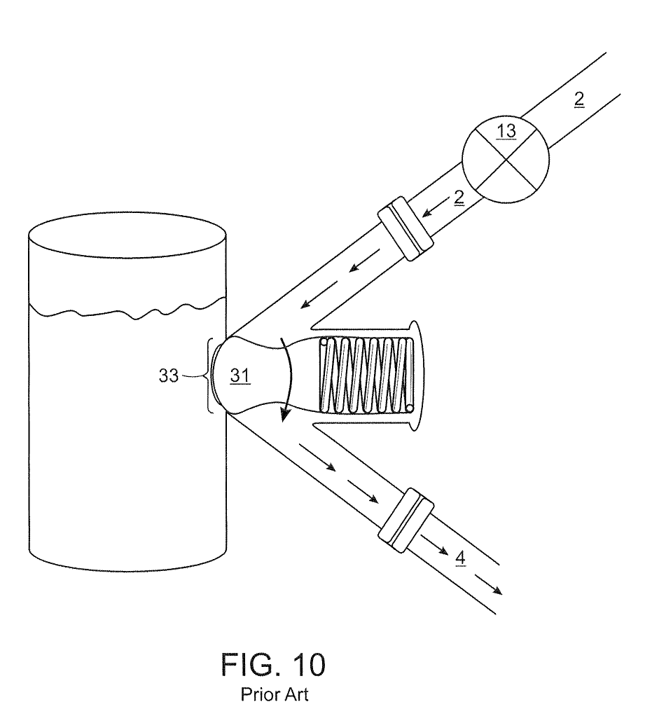

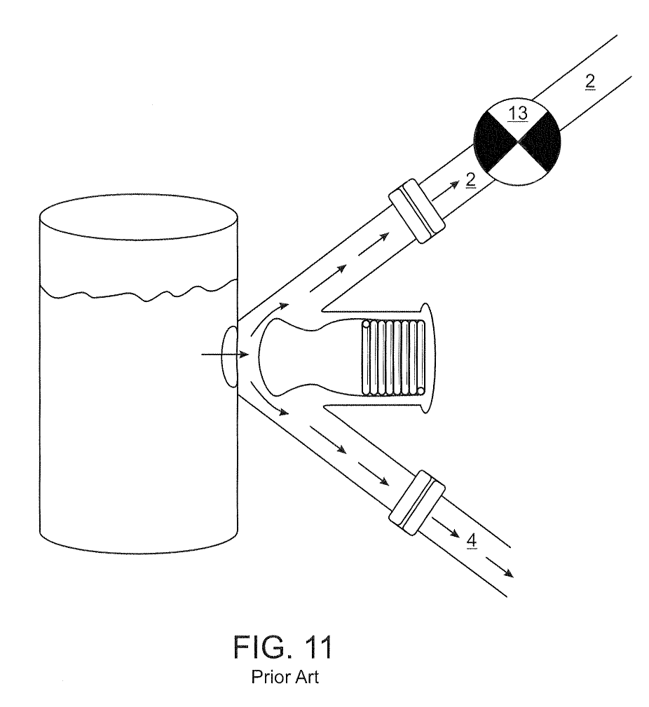

Sampling valve 3 is typically a three-port plunger valve specifically adapted for sterile sampling of a liquid sample from a container, such as the Keofitt® W15™ sampling valve, or the valves described in U.S. Patent Application Publication No. 2007/0074761 incorporated herein by reference in its entirety. An example of a suitable Keofitt® sampling valve is shown in FIGS. 10 and 11. Sampling valve 3 is connected to three components of the system: the steam channel 2, a fluid sample source 1, such as a reactor vessel, and a steam/sample channel 4. Steam and fluid samples can flow from the sampling valve 3 to isolation valve 17 through steam/sample channel 4. Steam/sample channel 4 typically has an inner diameter of about 9 mm. When closed as shown in FIG. 10, steam is able to flow from steam channel 2 to steam/sample channel 4. When opened, as shown in FIG. 11, fluid sample flows from port 33 toward steam/sample channel 4, the flow path toward steam channel 2 being blocked by steam valve 13.

Isolation valve 17 is typically a three-port diaphragm valve. An example of a suitable isolation valve is a GEMÜ® Type 650 TC TFE 15RaEP Con1, which is a ⅜ inch three-port pneumatically actuated sanitary valve. A first port of isolation valve 17 is connected to the steam/fluid channel 4, while a second port of isolation valve 17 is connected to drain channel 8 and a third port of the isolation valve 17 is connected to sample transfer channel 6.

Sample transfer channel 6 establishes fluid communication between isolation valve 17 and processing system 11. As used herein, “fluid communication” refers to a relationship between two components by which fluid can be permitted to flow from one component to the other. Processing system 11 can include cleaning, processing, and analytical instrumentation, as well as controller 27, which will be described further below. An example of a suitable processing system is described in U.S. Patent Application Publication No. 2004/0259266, incorporated herein by reference in its entirety. Processing system 11 further includes a cleaning fluid source 40, a sterile water source 30, and an internal valve 29, which opens and closes fluid communication to isolation valve 17.

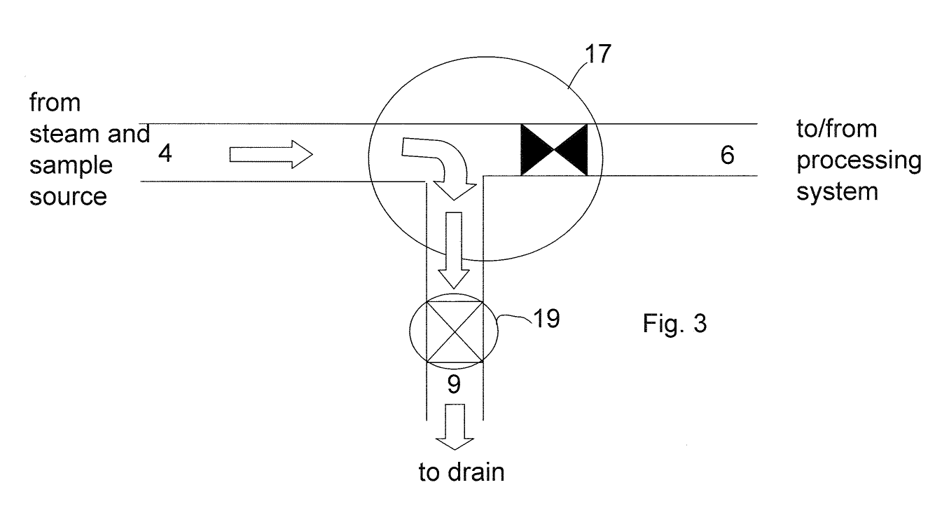

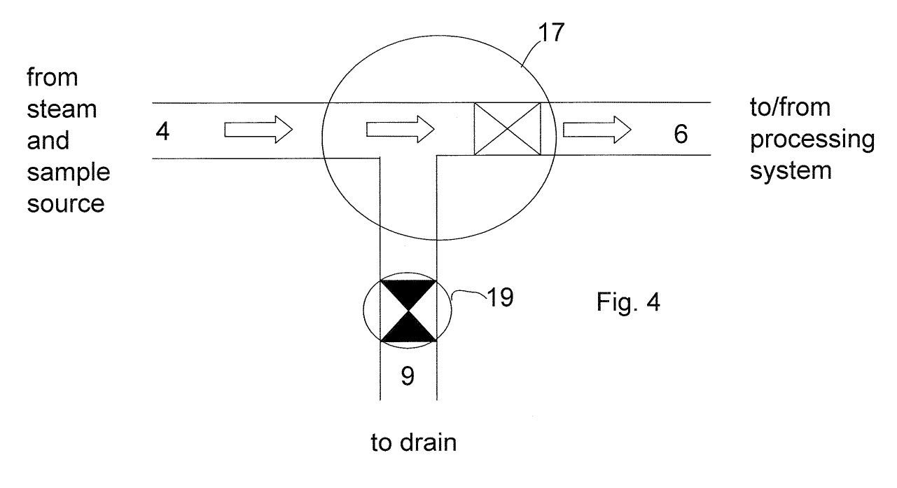

In one embodiment, the isolation valve 17 essentially operates in the manner shown in FIGS. 3, 4, 5a and 5b. In cooperation with the drain valve 19, isolation valve can pass steam and fluid samples to the drain as shown in FIG. 3, or pass fluid samples to a processing system as shown in FIG. 4. In FIG. 3, steam and fluid samples are prevented from entering sample transfer channel 6 and the processing system. In FIG. 4, fluid samples are allowed to pass to sample transfer channel 6 and enter the processing system. The sample fluid does not pass through drain valve 19, which is closed during a sampling operation.

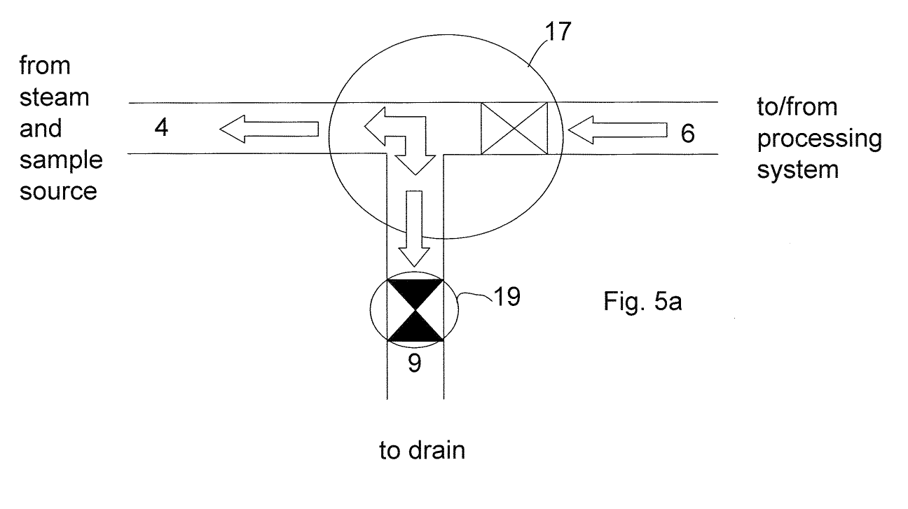

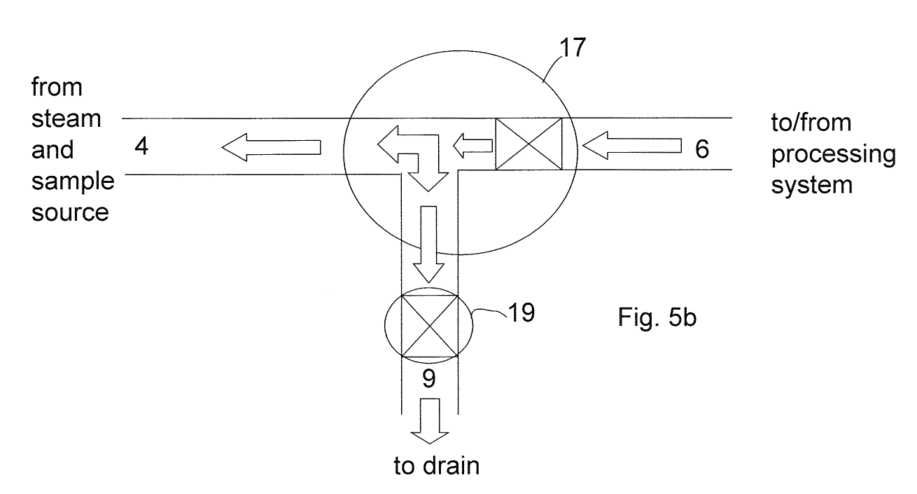

As shown in FIGS. 5a and 5b, isolation valve 17 also routes cleaning fluid from the processing system through sample transfer channel 6 to the drain. FIGS. 4, 5a, and 5b show that isolation valve 17 is in mutual fluid communication with the processing system via sample transfer channel 6. That is, fluid samples can be permitted to flow through isolation valve 17 to the processing system 11 as in FIG. 4, and cleaning fluid can be permitted to flow from the processing system 11 through isolation valve 17, as in FIGS. 5a and 5b.

The drain valve is typically similar to the isolation valve, but has two ports instead of three. An example of a suitable drain valve is a GEMÜ® Type 650 TC TFE 15RaEP Con1 having two ⅜ inch ports, which is also a pneumatically actuated sanitary valve. In the alternative, isolation valve can perform the above functions without the assistance of drain valve 19, so long as isolation valve is a true three-way valve, rather than a three-port valve with two ports always coupled together.

As shown in FIG. 2, the system may also include an optional manual sampling valve 15. Manual sampling valve is typically a three-port plunger valve, such as GEMÜ® Type 601 TC TFE 15RaEP Con A-B, which is a ⅜ inch three-port manually actuated sanitary valve. Manual sampling valve 15 is connected to an optional manual sampling output port 21, which can be used by a human operator to draw fluid samples from the fluid sample source 1. The manual valve operates in a similar manner as the isolation valve 17. However, during normal automatic operation, the valve shuts the fluid pathway to manual output port 21.

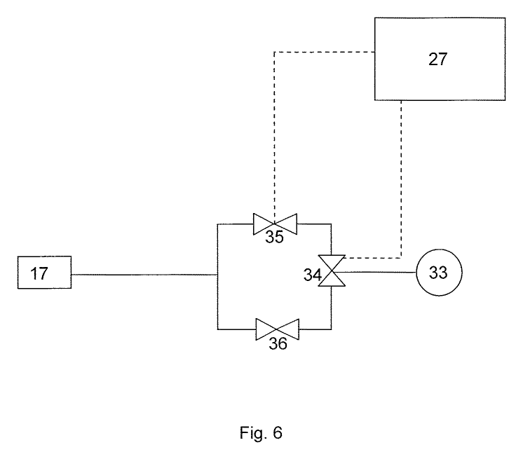

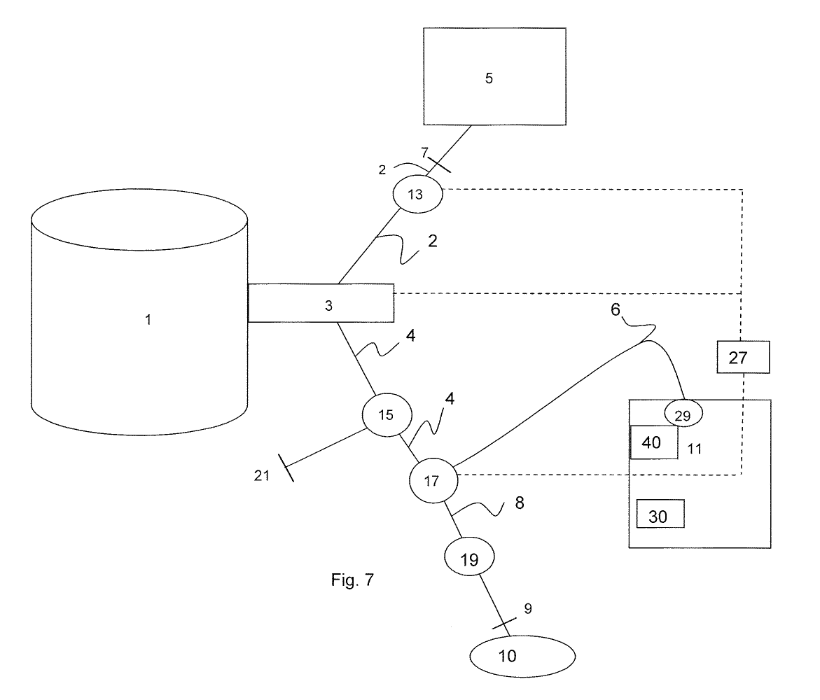

The steam valve 13, sampling valve 3, isolation valve 17, drain valve 19, and internal valve 29 are controlled in sequence to perform various system operations, which will be described in detail below. Each of the valves is pneumatically actuated by one of two control valves in parallel: a solenoid control valve and a manual control valve. For example, FIG. 6 shows isolation valve 17, which is pneumatically actuated by either manual control valve 36 or solenoid control valve 35. The user can select between automatic and manual control by toggling auto/manual solenoid switch valve 34, which is connected to compressed air source 33. The valve switches compressed air from source 33 to either the solenoid valve 35 for automatic control or manual control valve 36 for manual control. Under normal operation, the valves of the system are controlled automatically. A controller 27, such as a programmable logic controller (PLC) controls the solenoid valves and solenoid switch valves. As shown in FIG. 2, the controller typically resides in processing system 11 and controls the control valves to actuate the pneumatic valves, thereby automatically performing the various operations of the system in sequential order periodically throughout the bioreactor process. In one embodiment, such as the one shown in FIG. 7, the controller 27 is a separate component of the sampling system, and not part of the processing system 11.

### OPERATION OF THE SYSTEM

Before a new sample can be extracted from the reactor vessel, parts of the sampling system are sterilized, while others are sanitized. As used herein, the term “sterile” refers to a system or components of a system that are absolutely free of unknown living organisms or bioactive DNA. As thus defined, sterility has been proven by experiment to be achieved only by high temperature steam or radiation. As used herein, the term “sanitized” refers to a system or components of a system that are free of unknown organisms in measurable levels.

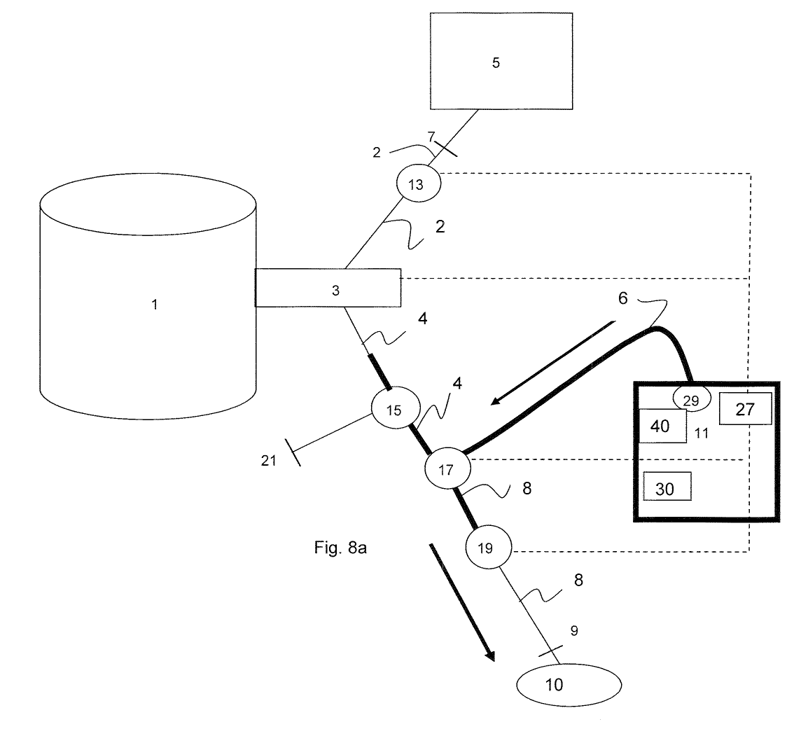

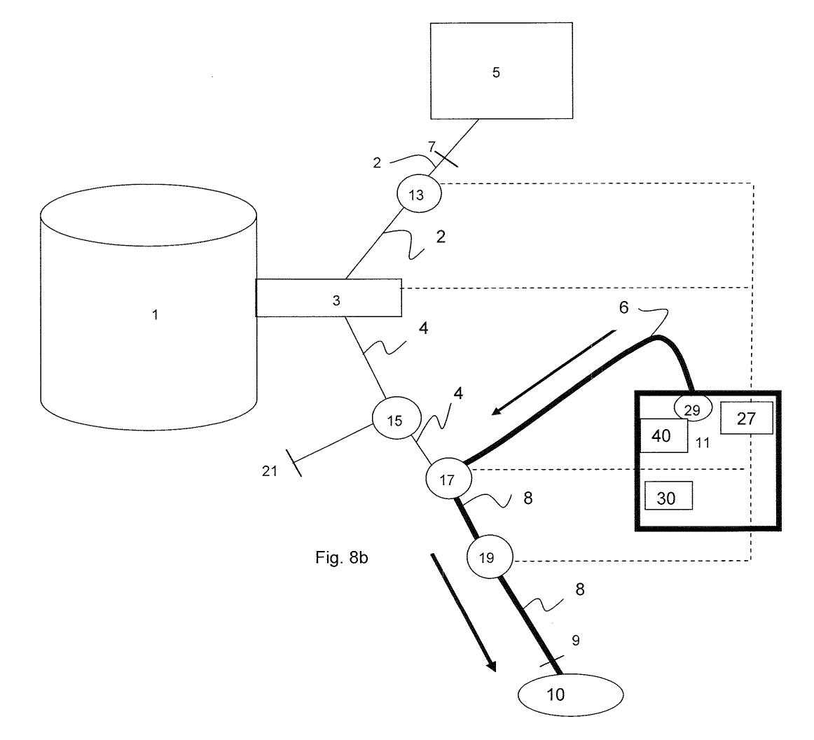

In the embodiment shown in FIGS. 8a and 8b, the sample transfer channel 6 is sanitized. Sanitizing sample transfer channel 6 ensures that any residual organisms that may exist in the sample transfer channel 6 from a prior sampling operation do not enter steam/sample channel 4 when steam/sample channel 4 and sample transfer channel 6 are in fluid communication, such as when isolation valve 17 permits a fluid sample to enter the sample transfer line 6 during a sampling operation, described further below.

Internal valve 29 opens to permit fluid to flow. For example, when internal valve 29 is open, cleaning fluid can flow from cleaning fluid source 40 through sample transfer channel 6 to isolation valve 17. As shown in FIG. 8a, drain valve 19 remains closed for the first part of the sanitizing operation. Cleaning fluid flows from the processing system 11, through sample transfer channel 6 and partially into steam/sample channel 4. Thus, the sample transfer channel 6, isolation valve 17, and a portion of steam/sample channel 4 are sanitized.

The second part of the sanitizing operation is shown in FIG. 8b. At this time, drain valve 19 opens so that cleaning fluid flows to the drain 10. The cleaning fluid flushes the isolation valve 17 and sample transfer channel 6 of any sample material remaining from the previous sampling operation. After the cleaning fluid has passed to the drain 10, internal valve 29 and drain valve 19 remain open to permit sterile water from sterile water source 30 to further rinse the sample transfer channel 6 and isolation valve 17 and exit the system via drain channel 8. At the end of the sanitizing operation, residual sterile water still remains in sample transfer channel 6.

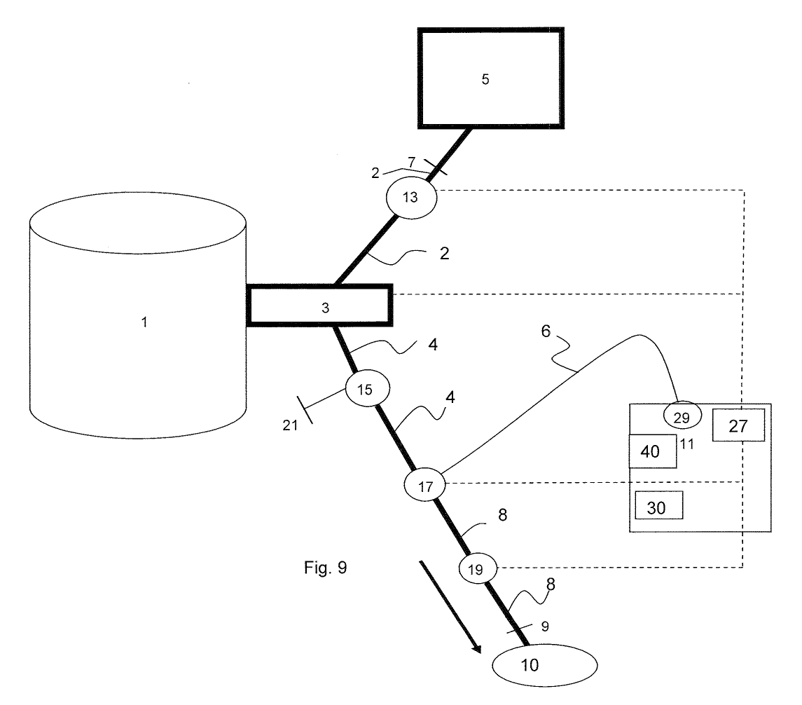

The system then undergoes a sterilizing operation, as shown in FIG. 9. Drain valve 19 remains open while internal valve 29 and isolation valve 17 are closed. As shown in FIG. 3, even when isolation valve 17 is closed, steam from steam/sample channel 4 is still permitted to pass to the drain 10. Thus, steam valve 13 is opened and steam passes from steam source 5 through steam channel 2, sampling valve 3, steam/sample channel 4, and drain channel 8 to the drain 10. Steam is allowed to flow for a specified duration and temperature that is sufficient to ensure sterilizing of sampling valve 3. The duration is typically at least about 20 minutes and the temperature of the steam is typically at least about 131 degrees Celsius. The steam pressure within the system during the sterilizing operation is greater than atmospheric pressure.

FIG. 10 shows the sterilizing of sampling valve 3 in detail. Valve head 31 is seated over an aperture 33, thereby obstructing the flow of fluid from fluid sample source 1. Steam enters sampling valve 3 from the steam source (not shown) through steam channel 2 and exits through steam/sample channel 4.

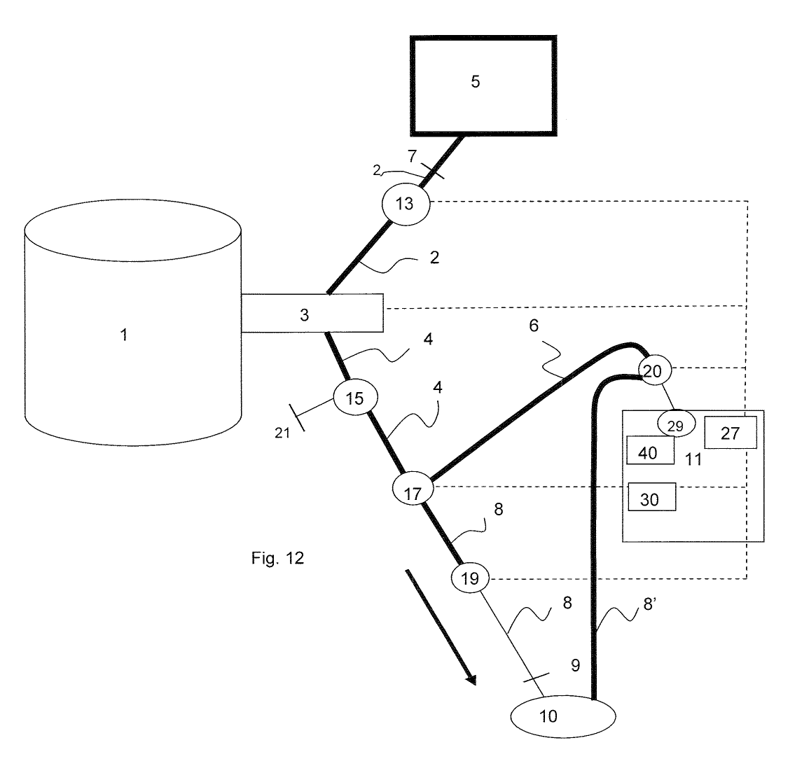

In one embodiment, sample transfer channel 6 can be at least partially sterilized. As shown in FIG. 12, a sample transfer valve 20 is positioned to allow steam to travel up the sample transfer channel 6 and to the drain 10 while blocking steam from reaching the heating processing system and causing damage to the electrical components. An additional drain channel 8′ is required for this embodiment. The sample transfer channel 6 for this embodiment preferably has an inner diameter that is greater than about 1 mm, in order to allow steam to pass through sample transfer channel 6. Most of the sample transfer channel 6 is sterilized.

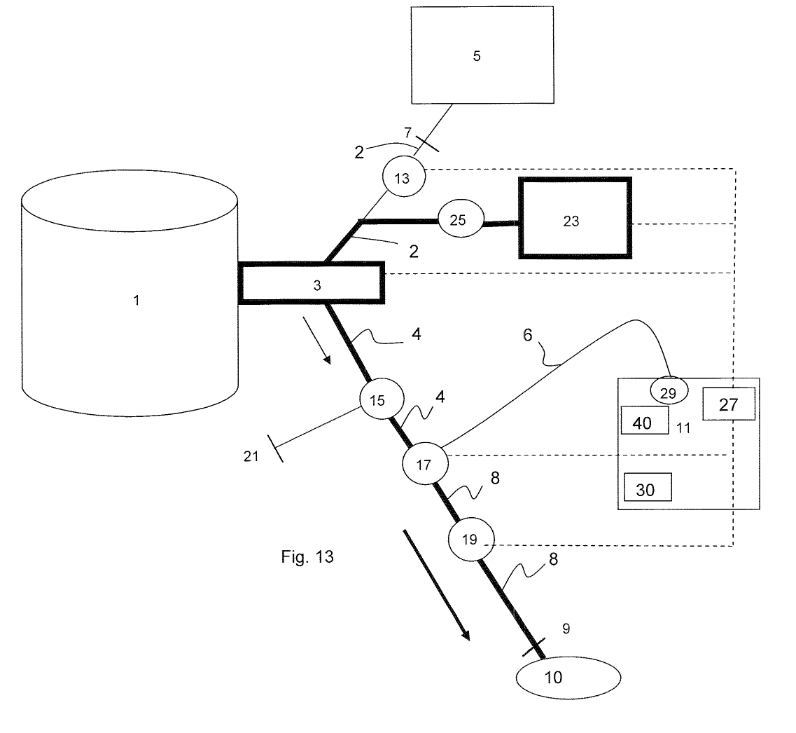

Once the sterilizing operation has completed, steam valve 13 closes and the system is sufficiently free of contamination. However, the system components generally remain hot from the sterilizing operation. To reduce the temperature of the components, the system can undergo an optional cooling operation, as shown in FIG. 13. In the cooling operation, an optional cooling valve 25, connected to an optional sterile air source 23, opens to allow sterile air to flush and cool the system components, particularly the sampling valve 3 and steam/sample channel 4. The sterile air is allowed to flow for a specified duration and temperature that is sufficient to ensure cooling of the sampling valve 3. Typically, the temperature of the sterile air is between about 15 to about 20 degrees Celsius. After the system has reached a temperature sufficient to allow a sample to be extracted from the reaction vessel, cooling valve 25 closes. This operation ensures that subsequent fluid samples, which are often proteinaceous, do not denature in the system.

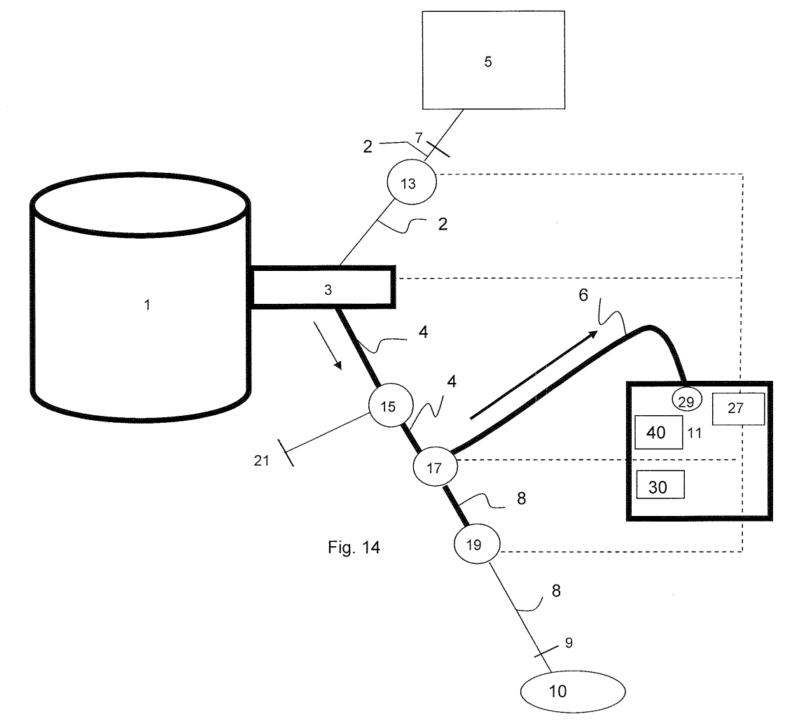

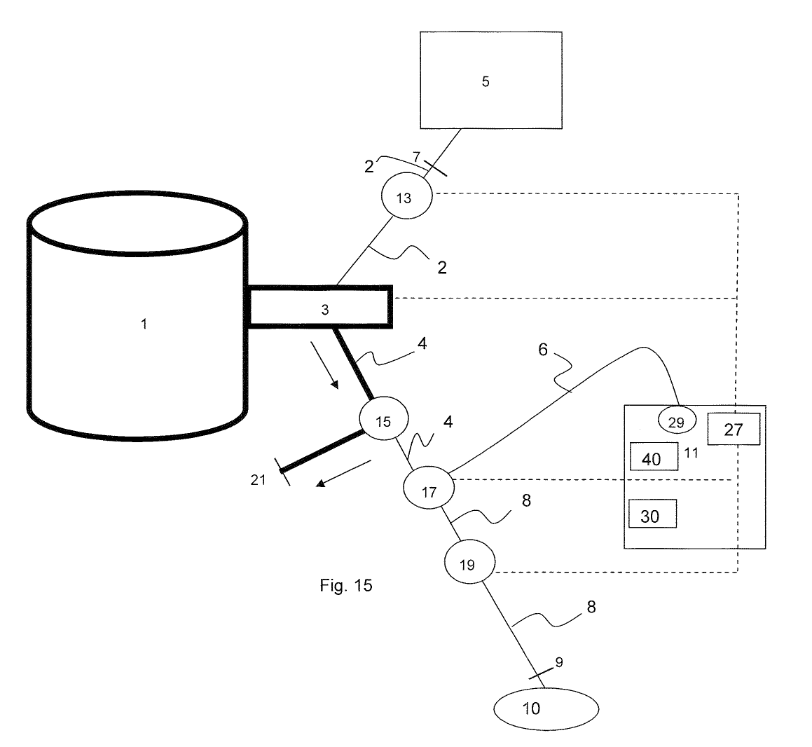

Immediately prior to the sampling operation, drain valve 19 closes so that fluid samples cannot flow to drain 10. As shown in FIG. 14, sample valve 3 isolation valve 17, and internal valve 29 are opened and a sample is allowed to flow from the fluid sample source 1, through the sampling valve 3, steam/sample channel 4, isolation valve 17, and sample transfer channel 6 into the processing module 11, where the sample may be processed and analyzed. FIG. 11 shows sampling valve 3 during the sampling operation in detail. Valve head 31 is removed from port 33 by pneumatic control and fluid is allowed to flow from fluid sample source 1 through steam/sample channel 4. The steam valve 13 along the steam channel 2 prevents fluid samples from flowing to the steam source. Alternatively, as shown in FIG. 15, a sample may be taken manually via manual sample valve 15, which routes the sample from sampling valve 3 to sample output port 21. Immediately subsequent to the sampling operation, the system may perform an additional sanitizing and sterilizing operation in the manner described above.

To ensure that the system extracts a sample for analysis that is representative of the batch in the sample source 1, steam/sample channel and sample transfer channel are “primed,” or flooded with sample fluid. That is, during the sampling operation, the system extracts more fluid than necessary to perform an analysis. For example, a total of 30 ml of the batch fluid is extracted from the sample source in order to obtain a 10 ml aliquot; the first 20 ml is primer to flush the fluid lines of residual fluid and the final 10 ml is the actual sample to be analyzed. This practice is typical for previously known manual systems as well as the presently described system, and it prevents the analysis sample from being diluted by residual fluid as it flows through the system. In contrast to previously known manual systems, the present automated system is capable of consistently and accurately providing the exact amount of fluid sample required to prime the fluid lines, thus minimizing waste of the sample.

In some embodiments, where even the smallest amount of the batch material is highly valuable, the dead volume of the sample transfer channel 6 is sized as small as possible to avoid drawing more fluid sample than is needed for analysis. Typically, the sample transfer channel 6 has an inner diameter between about 1 mm and about 2 mm, and a dead volume of less than about 60 ml. Thus provided is a safer, more consistent sterile sampling system that minimizes sample waste and performs sampling operations automatically.

Further embodiments particularly suited for automatic sampling of heterogeneous fluids, such as mammalian cell cultures, can be found in. U.S. Ser. No. 61/133,171, entitled, “Improved System and Method for Automated Sterile Sampling of Fluid From a Vessel,” of Erwin Yaokui Yu, Marcel J. Meacham and George E. Barringer, Jr., (Attorney Docket No. 3551.1014-000) which application was filed Jun. 25, 2008, and which application is incorporated by reference herein in its entirety.

The teachings of all patents, published applications and references cited herein are incorporated by reference in their entirety. While this invention has been particularly shown and described with references to example embodiments thereof, it will be understood by those skilled in the art that various changes in form and details may be made therein without departing from the scope of the invention encompassed by the appended claims.

What is claimed is:

1. An automatic sterile sampling system for sampling fluid samples from a sample source and providing the sample to a processing system, comprising:
a steam valve to receive steam from a steam source;
a fluid sample source;
a processing system to process fluid samples from the fluid sample source and comprising a cleaning fluid source;
a sampling valve to receive fluid samples from the fluid sample source and connected to receive the steam from the steam valve;
an isolation valve to pass steam from the sampling valve to a drain, pass fluid samples from the sampling valve to the processing system, and pass cleaning fluid from the processing system to the drain; and
a controller configured to control the valves to control the flow of the steam, the fluid sample, and the cleaning fluid.

2. The system of claim 1, further comprising a sterile air source and valve connected to receive sterile air from the sterile air source and connected to flush the sampling valve with sterile air.

3. The system of claim 1, further comprising a manual sampling valve connected to receive fluid sample from the sampling valve and pass fluid sample to a manual sampling output port.

4. The system of claim 1, wherein a dead volume of a fluid path between the isolation valve and the processing module is less than about 60 ml.

5. The system of claim 1, wherein the steam valve is connected to a steam source.

6. The system of claim 1, wherein the isolation valve is connected to a drain to receive the steam, the fluid sample, and the cleaning fluid.

7. The system of claim 1, further comprising a drain valve connected to a drain to pass the steam and the cleaning fluid.

8. The system of claim 1, wherein the processing system further comprises a sterile water source.

9. The system of claim 1, wherein the fluid path between the isolation valve and the processing module includes a sample transfer valve to prevent steam from entering the processing module.

10. A method for automatic aseptic sampling from a fluid sample source, comprising the steps of:
providing a steam source, a steam valve connected the steam source, a sampling valve connected to the fluid sample source, an isolation valve, a processing module, a drain valve, a drain, and a controller; and
employing the controller to:
pass cleaning fluid from the processing module through the isolation valve to the drain;
pass steam through the steam valve, sampling valve, and isolation valve to the drain for a duration sufficient to sterilize the sampling valve, the isolation valve, and a fluid path therebetween; and
pass fluid sample from the fluid sample source through the sampling valve and isolation valve to the processing module.

11. The method of claim 10, further comprising the step of employing the controller to pass sterile air through a sterile air valve, the steam valve, sampling valve, and isolation valve to the drain for a duration sufficient to cool the sampling valve prior to the step of employing the controller to pass fluid sample from the fluid sample source through the sampling valve and isolation valve to the processing module.

12. The method of claim 10, further comprising the step of employing the controller to pass sterile water through from the processing module through the isolation valve to the drain after the step of employing the controller to pass cleaning fluid from the processing module through the isolation valve to the drain.

13. The method of claim 10, further comprising the step of employing the controller to pass steam through at least a portion of a fluid path between the isolation valve and the processing system.

14. The method of claim 10, further comprising the step employing the controller to of pass cleaning fluid from the processing module through the isolation valve and at least a portion of a fluid path between the isolation valve and the sampling valve immediately prior to the step of employing the controller to pass cleaning fluid from the processing module through the isolation valve to the drain.
